(19) United States
(12) Patent Application Publication
(10) Pub. No.: US 2022/0218942 A1
(43) Pub. Date: Jul. 14, 2022

(54) FULL-SENSORY GUIDED-MEDITATION SYSTEM

(71) Applicant: Kevin Jain, Chicago, IL (US)

(72) Inventor: Kevin Jain, Chicago, IL (US)

(21) Appl. No.: 17/145,996

(22) Filed: Jan. 11, 2021

(51) Int. Cl. A61M 21/02

(52) U.S. Cl. CPC A61M 21/02; A61M 2021/0027

(57) ABSTRACT

A full-sensory, guided-meditation system operative to administer a themed, sensory-stimulation scheme associated with a simulated scenario so as to provide a meditative experience engaging all senses in accordance with user needs and preferences. The system is configured to autonomously improve the meditative experience as the quantity and the quality of available user data increases.

### BACKGROUND OF THE INVENTION

The present invention is an electronic, guided-meditation system operative to provide personalized, full-sensory meditative-experiences and measure resulting physiological changes.

Typically, systems directed to facilitating meditation provide stimulation of one or two senses. Some systems combine sense stimulation with physiological indicator sensing. However, these systems lack full-sensory stimulation, full physiological indicator tracking, sense stimulation that reflects the physiological state and user preferences.

Therefore, there is a need for a full-sensory, guided mediation system operative in accordance with a complete array of real-time physiological indicators and user preferences.

### SUMMARY OF THE INVENTION

According to the teachings of the present invention there is provided, a method for providing full-sensory guided meditation performed on a computer having a processor, memory, and one or more code sets stored in the memory and executed in the processor, the method including: administering a sensory stimulation scheme to at least one user, the stimulation scheme characterized by a themed set of sensory stimulations of one primary stimulation and a plurality of secondary stimulations; and capturing a plurality of physiological indicators of the user during administration of the sensory stimulation scheme.

According to a further feature of the present invention, the primary stimulation is implemented as visual imagery or audial content.

According to a further feature of the present invention, the plurality of secondary stimulations is implemented as two or more stimulations selected from the group consisting of audial stimulation, transcranial stimulation, olfactory stimulation, gustatory stimulation, haptic stimulation, and all combinations thereof.

According to a further feature of the present invention, the transcranial stimulation is implemented as transcranial direct-current stimulation or transcranial magnetic stimulation.

According to a further feature of the present invention, the plurality of physiological indicators is implemented as two or more indicators selected from the group consisting breathing rate, pulse rate, heart variation, electroencephalography (EEG) feedback, blood pressure, galvanic skin conductance, muscle movement, and combinations thereof.

According to a further feature of the present invention, there is also provided modifying the sensory stimulation scheme in accordance with at least one of the plurality of the physiological indicators.

According to a further feature of the present invention, there is also provided defining the sensory stimulation scheme in accordance with user preferences received from the user.

According to a further feature of the present invention, there is also provided defining the sensory stimulation scheme in accordance with physiological indicators shared by the user and a population having two or more common demographic features.

According to a further feature of the present invention, there is also provided defining the sensory stimulation scheme in accordance with physiological indicators shared by the user and a population having three or more common demographic features.

According to a further feature of the present invention, there is also provided outputting the physiological indicators to an output device or a dedicated user profile data base.

There is also provided according to the teachings of the present invention, a full-sensory, guided-meditation system, the system including: a set of three or more sensory stimulators of one primary stimulator and a plurality of secondary stimulators; a computer configured to: administer a sensory stimulation scheme to at least one user, the stimulation scheme characterized by a themed set of sensory stimulations of one primary stimulation and a plurality of secondary stimulations; and a set of physiological sensors configured to capture a plurality of physiological indicators of the user during administration of the sensory stimulation scheme.

According to a further feature of the present invention, the plurality of secondary stimulators is selected from a group consisting of a video display, ear phones, an olfactory stimulator, a gustatory stimulator, a haptic stimulator, a transcranial stimulator, and all combinations thereof.

According to a further feature of the present invention, the set of a plurality of secondary stimulators contains ear phones, an olfactory stimulator, and a gustatory stimulator.

According to a further feature of the present invention, the transcranial stimulator is implemented as a transcranial direct-current stimulator or transcranial magnetic stimulator.

According to a further feature of the present invention, the set of physiological sensors includes a plurality of sensors selected from the group consisting of a breathing rate sensor, a pulse rate sensor, a blood pressure sensor, a galvanic skin conductance sensor, a muscle movement sensor, a heart variation sensor, an electroencephalograph, and combinations thereof.

According to a further feature of the present invention, the set of physiological sensors contains a breathing rate sensor, a pulse rate sensor, and a blood pressure sensor.

According to a further feature of the present invention, the computer is further configured to modify the sensory stimulation scheme in accordance with at least one of the physiological indicators captured by the set of sensors from the user.

According to a further feature of the present invention, the computer is further configured to define the sensory stimulation scheme in accordance with user preferences received from the user.

According to a further feature of the present invention, the computer is further configured to define the sensory stimulation scheme in accordance with a population having two or more common demographic features with the user.

According to a further feature of the present invention, the computer is further configured to output the physiological indicators to an output device or a dedicated user profile data base.

### BRIEF DESCRIPTION OF THE DRAWINGS

The subject matter regarded as the invention is particularly pointed out and distinctly claimed in the concluding portion of the specification. The invention is best understood in view of the accompanying drawings in which:

FIG. 1A-1C are various, schematic views of a full-sensory meditation system, according to an embodiment;

FIG. 2 is a schematic view of user undergoing a guided meditation experience with the system of FIGS. 1A-1C, according to an embodiment;

FIG. 3A is block diagram of hardware and software components of the full-sensory meditation system, according to an embodiment;

FIG. 3B is sample user profile including user-supplied, demographic data and preferences, baseline physiological indicators, and target physiological indicators, according to an embodiment;

FIG. 4 is flow diagram of processing steps employed by the full-sensory meditation system, according to an embodiment;

FIG. 5 is a sample stimulation scheme of the full-sensory meditation system during a guided mediation, according to an embodiment;

FIG. 6 is a sample physiological indicator profile of the user during administration of the stimulation scheme of FIG. 5, according to an embodiment;

FIG. 7 is a comparison of a galvanic skin response profile and a target value according to an embodiment;

FIG. 8 depicts of series of correlations between galvanic skin response and other stimulation types, according to an embodiment;

FIG. 9 depicts of series of correlations between galvanic skin response and variant olfactory stimulations, according to an embodiment;

FIG. 10 depicts an adjusted stimulation scheme directed to achieving the target galvanic skin response of FIG. 7; and

FIG. 11 is a plot of a relaxation index as a function of time characterizing the effectiveness of the full-sensory meditation system.

It will be appreciated that for the sake of clarity, elements shown in the figures may not be drawn to scale and reference numerals may be repeated in different figures to indicate corresponding or analogous elements.

### DETAILED DESCRIPTION OF THE PRESENT INVENTION

The following detailed description sets forth various details to facilitate understanding of the invention; however, it should be understood by those skilled in the art that the present invention may be practiced without these specific details. Furthermore, well-known methods, procedures, and components have not been omitted to highlight the invention.

The present invention is a full-sensory, meditation system operative to provide of personalized, themed stimulation schemes of various settings scenes. The meditation system stimulation scheme administers up to six different general stimuli types of sensory stimulation scheme of varying administration parameters through a set of stimulators to generate sensorially-immersive, meditative experiences that engage, stimulate, and relax the user. In such immersive meditative experiences, a person can experience deep physiological relaxation through a perceived journey complete with various sensory sensations that engage, stimulate, and relax the nervous system. Each additional stimulator contributes to the user perception that the simulation is realistic.

The themed stimulation scheme is defined and later modified, at least in part, on the basis of physiological indicators captured by a sensor set prior to administration of the experience and then during administration of the experience to advantageously construct and modify the stimulation scheme in accordance with user physiology and desired physiological targets as will be further discussed.

Generally, the system is operative to use baseline physical and physiological indicators either measured from the user or obtained from a populace of similar demographics, sets physiological indicator targets, sets a trajectory, and during administration modifies the stimulation scheme dynamically to achieve the target physiological indicator targets.

The ability to achieve these physiological indicator increases with repeated meditative experiences because user physiological responsiveness is tracked and added to the user profile and statistical inferences drawn through machine learning techniques to generate more effective user-specific, meditative experiences in the future.

Session data of the meditative experience is output so the user and relevant clinician or qualified, user-approved party can evaluate session results to evaluate the efficacy of a given simulation against a given set of physiological indicators.

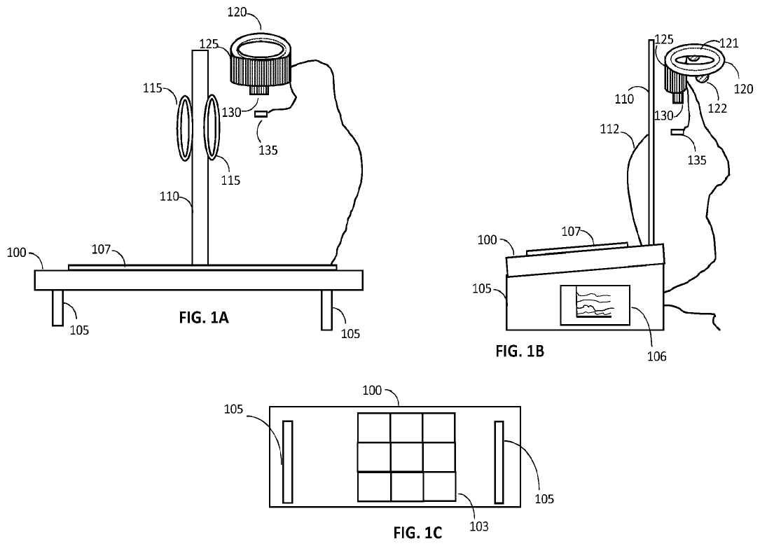

Turning now to the figures, FIG. 1A-1C are front, side, and bottom views, respectively, depicting a non-limiting example of a physical configuration of the full-sensory, meditation system. Specifically, a meditation bench includes a sitting platform 100, supporting elements 105 having a height enabling one sitting on platform 100 to fold legs underneath, a support column 110, and posture support 112, shoulder straps 115 and a set of sensory stimulators and another set of sensors.

The sensory stimulators include, include a brain wave emitter 121, a visual stimulator 125 implementable as virtual reality glasses or augmented reality glasses, earphones or earbuds 122, an olfactory stimulator 130, a taste stimulator 135, and haptic stimulator 107.

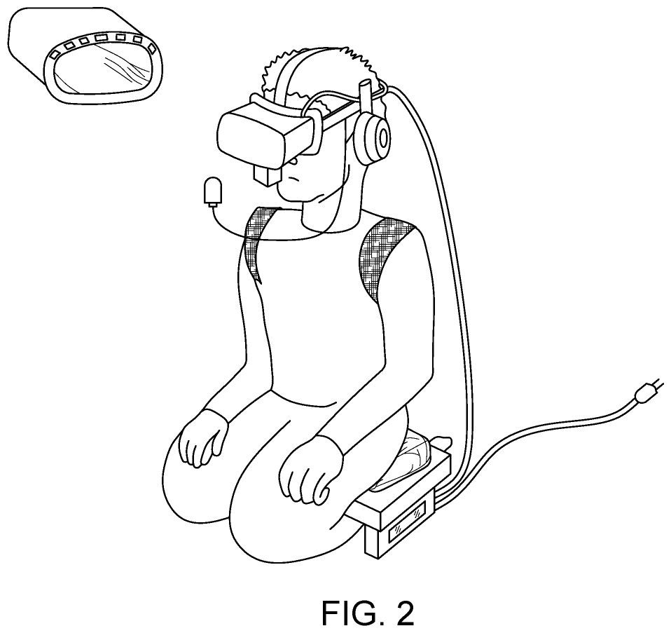

The set of sensors include electroencephalogram (EEG) sensors embedded in headband 120 (shown in FIGS. 1A-1B), electrocardiogram (EKG), respiratory sensor, galvanic skin response (GSR) sensor, blood pressure sensor, muscle movement sensor, and heat sensor all most clearly in the block diagram of FIG. 2. All stimulators and sensors are linked to a computer embedded in support structures, for example. Sensor output is displayed in display 106 and a touch screen 103 mounted to the underside of sitting platform 100 is operative to received user input to choose a guided experience in addition to configuration guidelines, preferences, other personal profile data.

It should be noted, that the system is operative to provide multiple combinations of sensory stimulation in given meditative experience in accordance with the user configured embodiment or in accordance with user needs and preferences found in the user profile. These combinations relate to the stimulation type administered, their sequence order, their respective time duration, and their respective intensity. For example, regarding stimulation types, in one embodiment the system administers either visual or audial stimulation as a primary stimulation and any combination of a plurality of two or more secondary stimulations. Secondary stimulations are audial when not used as a primary stimulation, olfactory, gustatory, haptic, and brain as will be further discussed. Each possible combination of the sensor stimulators embodies a different embodiment of the present invention.

Analogously, various combinations of three or more physiological sensors are employed in accordance with configuration choices. Each possible combination of three or more sensors embodies a separate embodiment of the present invention.

FIG. 2 is a schematic view of user engaged in a guided meditation with the system of FIGS. 1A-1C. As shown, the user is kneeling on a dedicated electronic bench 100. It should be appreciated that the system can be employed in a variety of postural support furniture like a bed, a chair, a hammock, a meditation pod, or other furniture facilitating user comfort and meditative attentiveness.

The system is deployable in homes, dedicated meditation studios, massage parlors for augmenting a massage experience, gyms for augmenting physical workouts with a rich mental component, office spaces, libraries, airports, malls, and even stadiums for mass communal relaxation events.

The system is also deployable in multiple, network-linked remote locations for distributed mass communal meditation sessions.

The system also facilitates group meditation to augment the effectiveness of the meditation experiences. Group meditation has been shown to have potentially synergistic effects. Such group meditations sessions can advantageously provide solace and group unity of teams, cities, communities in the face of tragedy, like terrorist attacks, war, and natural disaster.

The system is operative to utilize simulation scenarios independently developed and that have been shown to be statistically significant for various conditions. A few examples of various meditation scenarios are simulations directed to “focus before work”, “reduce depression”, “help with a grief cycle”, “overcome Post-Traumatic Stress Disorder (PTSD)”, “improve lucid dreaming”. Statistical significance can be tracked through anonymized data collection en masse through an anonymized profile, in a certain embodiment.

In a certain embodiment, the system is modular to facilitate addition, removal, and replacement of stimulators and sensors. Furthermore, the system is configurable to enable a user to define the type of sensory stimulations to be administered and those not to be administered in any given meditation session.

The full-sensory, guided meditation system has application for many segments of society like medical patients needing to relax, soldiers needing to relax, students wanting to relax, and laborers who want to relax, just to name a few. Furthermore, the system has utility for those suffering from PTSD, Anxiety, Depression, and many psychological and psychiatric areas.

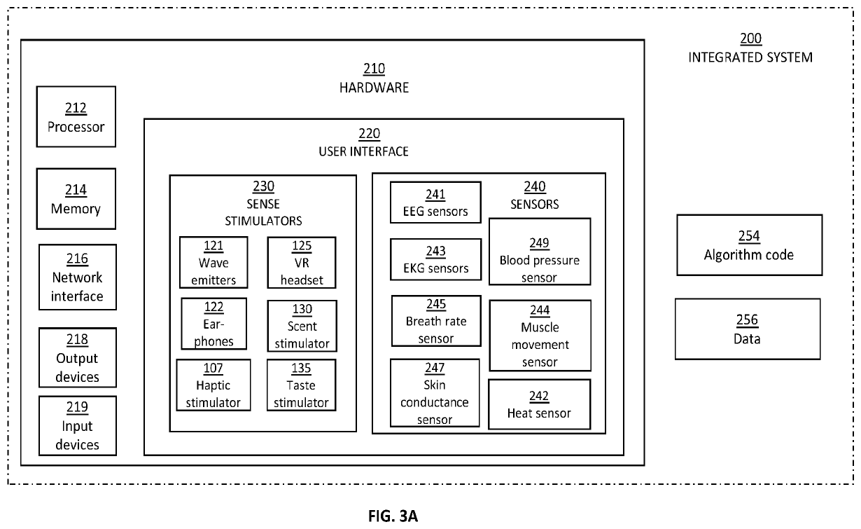

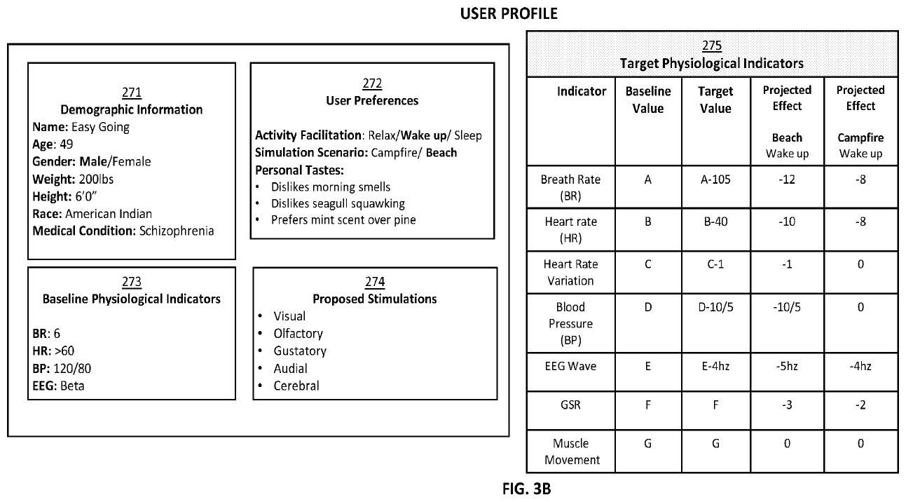

FIG. 3A is a block diagram of a full-sensory guided meditation system 200 including hardware 210 and software 250 components employed by the system, according to an embodiment.

Specifically, hardware 210 includes computer operational hardware of one or more linked processors 212, short- and long-term memory 214, a hard wired or wireless network interface 216, one or more output devices 218 like screens and printers, for example, and one or more input devices 219 like a keyboard, mouse, a microphone for voice activation of various simulation scenarios.

As previously noted, the user also interfaces with the meditation bench by way of a set of sense stimulators 230 operative to provide themed sensory stimulation associated with a chosen guided experience theme and sensors 240 for measuring physiological indicators. Presented here in the context of hardware interfacing a computer are stimulators 230 that include the above noted brain wave emitter 121, virtual reality (VR) headset 125 operative to display content of a chosen guided experience, earphones or earbuds 122, olfactory stimulator 130, taste stimulator 135, and haptic stimulator 107.

Sensors 240 include EEG sensors 241 for detecting brain wave activity, EKG sensors 243 for detecting heart variation and pulse rate, breath rate sensor 245, skin conductance sensor 247, blood pressure sensor 249, muscle movement sensor 244, and heat sensor 242.

Brain wave emitter 121 is operative to emit any one or a combination of wave frequencies associated with delta waves (0.5 to 3 Hz), theta waves (3 to 8 Hz), alpha waves (8 to 12 Hz), beta waves (12 to 38 Hz), and gamma waves (38 to 42 Hz).

VR headset 125 is implemented as the Oculus Rift variety, or other types providing stereoscopic virtual reality, in a certain embodiment. Scent emitter 130 is implemented as a Vasqo VR type or other types providing such functionality, in a certain embodiment

Taste stimulator 135 is implemented as a Brainport v100 or other stimulators providing such functionality. In another variant embodiment, a taste emitter is employed.

EEG sensors 241 are implemented in headband similar to that of the brain sensing headband of Muse™ available at https://choosemuse.com/, for example.

EKG sensors 243, are used to detect pulse rate and heart rate variability (HRV), according to an embodiment. In variant embodiments of the system, dedicated pulse tracking technology is employed.

Breath rate sensor 245 is implemented as any of a variety of respiratory rate sensor technologies like the non-contact sensor of XeThru found at www.xethru.com/respiration-monitoring.html or contact sensor of PMD found at http://www.pmd-solutions.com.

Skin conductance sensor 247 is implemented as galvanic skin detector measuring changes in skin conductivity like that available at IMOTIONS, found at 141 Tremont Street, 7th Floor, Boston, Mass., for example.

Blood pressure sensor 249 is implemented as an optical blood pressure sensor in a certain embodiment a non-optical sensor operative to render the blood pressure into a digital signal. An optical blood pressure sensor is available from Vicardio found at www.vicardio.care.

Muscle movement sensor 244 is implemented as a Electromyography (EMG) sensor available at Somaxis at https://www.somaxis.com.

In a certain embodiment multiple sensors are combined into a single device like an ExG Sensor that includes sensing for EEG, sEMG for muscle tension, and ECG for heart rate at Mindmedia at https://www.mindmedia.com or the Cricket device that includes sensing for muscle movement through) surface electromyography (sEMG), heart rate (EKG), brain activity (EEG), Posture (Gyr), respiration (Acc), Movement (Acc) also available at Somaxis at https://www.somaxis.com.

Heat sensor 242 is implemented as a Maxim Integrated MAX30205 Human Body Temperature Sensor available at Maxim Integrated found at Maximintegrated.Com or other temperature sensors providing such functionality. It should be appreciated that in another embodiment, posture and balance sensors are added to the system to provide additional insight into user response.

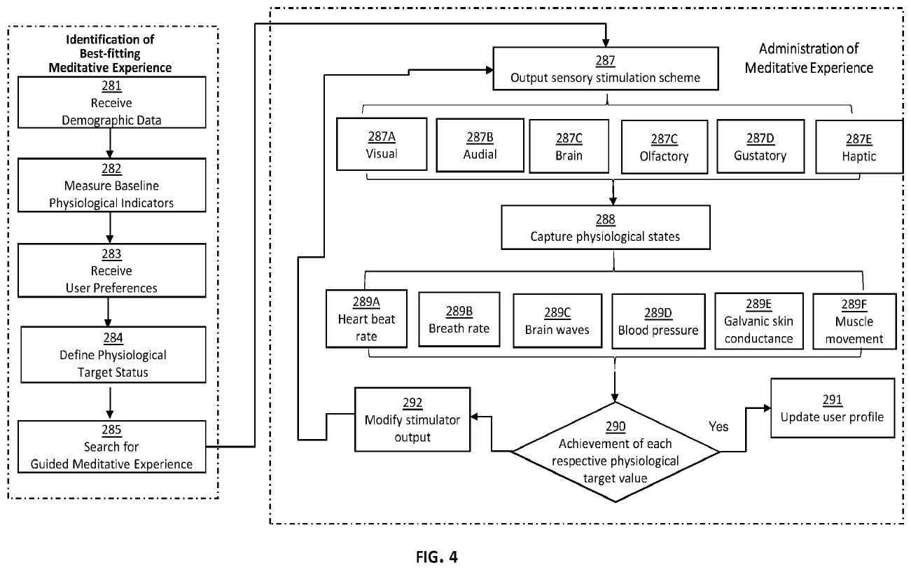

As shown, integrated system 200 includes code 254 and data 256 for implementing the guided experience. Code 254 includes the code used to implement the processing steps in FIG. 4 in addition to various machine learning techniques to construct the stimulation scheme prior to its administration and its modification during or after administration. As previously noted, the stimulation scheme is constructed in accordance with user profile data included in data 256 in addition general populace data relevant to the user profile. Also, data 256 includes data obtained from large trials and pervasive testing of the equipment and experiences of a meditating population. Such robust data improves data quality and advantageously increase the likelihood of accurately identifying proper correlations between the stimulation type and the result yielded.

FIG. 3B is a sample user profile including demographic information supplied to the system, measured baseline physiological indicators, choice of the activity to be facilitated through the guided experience, meditation style preferences, and personal tastes.

User demographic information 271 includes, inter alia, gender, age, weight, height, racially associated propensities, medical conditions including physical, emotional, or mental health issues, or other user conditions that should be considered when tailoring the most effective, guided meditative experience.

User preferences 272 refer to user choices, sensitivities, and idiosyncrasies in relation to the sensory stimulations and their application parameters like duration, intensity, and other relevant parameters. User preferences also includes target physiological indicators for any one or combination of the physiological indicators.

Such flexibility advantageously enables users to select the simulation scenario and within that context choose the scene variations that facilitate the chosen activity of endeavor, set physiological indicator goals, and also tailor the experience in accordance with personal sensory preferences. The system is operative to recommend meditative experiences that have historically succeeded in achieving target physiological states for either a general populace or individual users of the same demographic group in the absence of user physiological indicators. Subsequent user choices and stimuli responses are used by the system to upgrade the user profile and through machine learning enhance future meditative experiences.

Activity facilitation refers to the specific activity for which the user chooses to facilitate through the guided meditative experience. Sample activities include, inter alia, ‘Relax’, ‘Wake-up’, and ‘Prepare for Bed’ It should be appreciated that each activity may have a different user-specific, target value for each respective physiological indicator.

Simulation scenario refers to the scene or the setting simulated in the guided meditative experience. In a certain embodiment, setting choices include “Beach”, “Space”, “Campfire”. The system is configurable to further provide variation in each simulated settings to facilitate the chosen activity of endeavor. For example, a beach scene can be simulated as a sleepy beach scene to facilitate preparing for bed or an active beach scene simulated to facilitate waking up. Such variation is achieved through application of the appropriate sensory stimulation in the scene simulation. It should be appreciated that the system also has provisions enabling users to design experiences of different scenarios and the associated themed stimulation scheme.

Baseline physiological indicators 273 refer to any one or more of the above noted indicators measured prior to commencement of a meditative session to establish a starting point in the formulation of a stimulation scheme and track changes in physiological user state during administration of the stimulation scheme.

The measured values of the physiological indicators are assigned a status based on user-specific status thresholds defined on the basis of cumulative user data. If personalized physiological data is unavailable, the physiological indicator status is set on the basis of general demographic data and updated in accordance with personalized user data as it becomes available, in a certain embodiment. Sample physiological indicators status flags are set forth below:

Sample Physiological Status Targets

| | Relaxation | Wake-up | Sleep | Creative |
|---|---|---|---|---|
| Pulse | Low- | Average- | Low- | Low-high |
| rate | average | high | average | |
| Breath rate | Low | Average | Low | Low-high |
| EEG | Delta- alpha | Alpha-theta | Delta-theta | Theta |
| GSR | Low | Average | Low | Average- |
| | | | | high |
| Heart- rate | Low | Average | Low | Low-high |
| variation | | | | |
| Blood pressure | Average | Average | Low- | Low-high |
| | | | average | |
| Muscle | Average | Average | Average | Low-high |
| movement | | | | |

It should be appreciated that personalized nature of the physiological indicator status means that physiological indicator measurement for a first user can be rated as low and for a second user rated as average, for example.

Target physiological indicators 275 characterize the physiological indicator value for which the meditative experience strives to achieve given the baseline physiological indicators values, meditation scenario, the projected influence of the meditation scenario for each physiological indicator associated with a user.

The noted effects are user specific and therefore derived from past user experiences, in a certain embodiment. In the absence of data regarding the effect of a chosen meditative experience on a user, the stimulation scheme of the meditative experience is constructed on the basis of data associated with a population of shared demographics like common gender, common age group, common baseline indicators within a predefined range, and physiological state, for example. It should be appreciated that the degree of demographic commonality defining a demographic cohort is a configurable matter. Following is an example of demographic data that can be used to either construct a new stimulation scheme or identify an existing stimulation scheme:

| | | | Median State After |
|---|---|---|---|
| | | Median State After | “Relaxing Beach |
| | | “Relaxing Beach | Scene with Real |
| Measurement | ‘Before’ | Scene” | Time Adjustments” |
| Pulse | ‘Very high’ | ‘low’ | ‘low’ |
| Breath Rate | ‘High’ | ‘low’ | ‘Very low’ |
| EEG Waves | ‘Gamma’ | ‘alpha’ | ‘theta’ |
| GSR | ‘Very high’ | ‘Very high’ | ‘moderate’ |
| HRV | Any | Any | ‘Average’ |
| BP | Any | Any | ‘Average’ |
| Muscle | Any | Any | ‘Average’ |
| Movement | | | |
| Characterized | ‘Anxious’ | ‘Relaxed’ | ‘Relaxed’ |
| Physiology | | | |

FIG. 4 is flow diagram of processing steps employed by the full-sensory meditation system, according to an embodiment. As shown, processing steps include two stages; experience identification and experience administration.

Experience identification is directed to identifying the most appropriated guided, meditative experience in view of the user demographics, physiological indicators, goals and preferences. Specifically, in step 281 demographic data is received from the user or other relevant party on behalf of the user. The content included in demographic data is set forth above in the context of FIG. 3A.

In step 282, baseline physiological indicators are captured through the above noted sensors prior to the experience to set a baseline physiological state.

In step 283, the system receives user preferences relating the activity to be facilitated through the medicative experience, the simulation scenario, tastes of other idiosyncrasies to any one or combination of the stimulation types directly from the user or from the user profile.

In step 284, physiological, target status is received for each relevant physiological indicator. Physiological target status is either defined by the user if he is capable of doing so or obtained from a general database of ideal physiological indicators for individuals of common demographics involved in the chosen activity of endeavor. Alternatively, the system is configured to define target physiological indicators on the basis of past performances documented in the user profile. In another embodiment, the system is operative to In step 285, the system identifies a full-sensory, guided meditative experience from a search of a bank of stimulation schemes in accordance with the above-noted user demographic data, physiological characterization, preferences, and goals. The chosen meditative experience is characterized by projected physiological indicator values most closely matching the above-noted physiological target values. According to system configuration, the comparison between the target and projected indicator values is implemented either collectively or on the basis of one or more designated individual indicators. The projected target indicator value for each indicator is determined on the basis of known or hypothesized correlations between each stimulation type and physiological result as will be further discussed.

After the stimulation scheme for full-sensory, guided meditative experience has been constructed or identified, system processing proceeds to the second stage of the process to administer the stimulation scheme. During administration, the system tracks user response and dynamically adjusts the scheme responsively to a measured physiological indicator appearing to be off track from the target values as will be further discussed. User responsiveness is used to update the user profile to improve construction of tailored stimulation schemes in future sessions and to more effectively modify them. This updated, real-time sensory information creates a feedback loop which is checked against the expected trajectory of the experience. If the user is responding the stimuli in the expected fashion, or within the margin of acceptable error, the system proceeds as planned. However, when a threshold of significant deviation is detected, the system initiates a modification process of the stimulation scheme as will be further discussed.

Specifically, in step 287 the system administers combination of the above-noted sensory stimulations in a scheme in accordance the constructed or identified scheme associated with the simulation scenario. Sensory stimulations include primary stimulations of visual 287A stimulation and secondary stimulations including audial stimulation 287B, and additionally, brain stimulation 287C, olfactory stimulation 287D, gustatory stimulation 287E and haptic stimulation 287E. The stimulation scheme defines timing and duration of each type of stimulation in addition to intensity levels and other output-specific parameters like frequency, for example. In step 288, the system captures various physiological indicators through the above-noted sensors in contact with the user for the capture of heart beat rate 289A, breath rate 289B, EEG waves 289C, blood pressure 289D, galvanic skin conductance 289E, and muscle movement 289E. The EEG waves 289C can vary in frequency; delta waves of a frequency of 3 Hz or below, theta waves of a frequency of 3.5 to 7.5 Hz, alpha waves of a frequency between 7.5 and 13 Hz, beta waves of a frequency of 14 and greater Hz. In step 290 captured physiological state values are compared with the respective target values obtained either from the art or from a personalized profile. If the values achieve the respective target value, in step 291 the system updates the user profile to facilitate duplicability. If the target value has not been achieved, in step 292 the relevant stimulations are modified and re-administered in step 287 as will be further discussed.

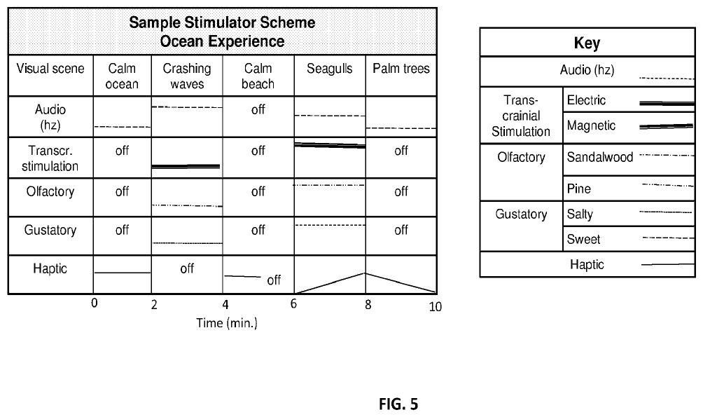

FIG. 5 is a sample stimulation scheme of the full-sensory meditation system during a guided ocean experience employing two-minute intervals. As shown, the primary visual content is displayed continuously and the secondary stimulation types augment the visual experience in accordance with configuration guidelines.

As shown, the guided experience begins with a calm ocean visual scene and continues for two minutes, then changes to a wave crashing scene for two minutes, at four minutes a calm beach scene is presented for two minutes, then a seagull scene for two minutes, and then at eight minutes a palm tree scene is presented until the end of the guided experience at ten minutes. It should be appreciated that the total duration of the experience is configurable as well the time intervals for each scene. As previously noted, additional sensory stimulations augment the immersive capacity of the guided experience. The stimulation scheme is tailored in accordance with user profile data and known or hypothetical correlations between the physiological indicators to achieve a chosen physiological state. As shown, visual content is presented as the primary stimulation to which other stimulation types augment. However, it should be appreciated that this is a configuration choice and that any of the specific stimulation types can be designated as a primary stimulation to which other secondary stimulation types enhance and deepen. Primary stimulation is characterized by content presented independently to the other stimulation types. In contrast, secondary stimulation is characterized by its dependency on content of the primary stimulation.

Continuing now with a sample specific stimulation scheme augmenting the primary visual stimulation based on two-minute intervals:
Binaural audial stimulation commences at the beginning of the guided experience at low-volume, increase to high volume, ceases entirely at four minutes, commences at six minutes at a medium volume, and continues at eight minutes at low volume. It should be noted that non-binaural, audial stimulation is employed in combination with binaural, audial stimulation or in non-combination; in accordance with configuration parameters.
Magnetic transcranial stimulation commences at two minutes as low-intensity, electric stimulation, ceases at four minutes, resumes at six minutes as high-level electrical stimulation, and ceases at eight minutes until the end of the guided experience.
Olfactory stimulation of low-concentration pine scent commences at two minutes, ceases at four minutes, resumes at six minutes as high-concentration pine scent, and ceases at eight minutes until the end of the guided experience.
Gustatory stimulation commences at two minutes as medium-concentration salt flavor, ceases at four minutes, resumes at six minutes as high-concentration sweet flavor, and ceases at eight minutes until the end of the guided experience.
Haptic stimulation commences at the beginning of the guided experience, ceases at two minutes, resumes at four minutes as high-intensity stimulation for one minute, at six minutes resumes as medium-intensity stimulation progressing to medium-intensity stimulation at eight minutes and then progressively decreasing in intensity to no stimulation at the end of the guided experience.

It should be appreciated that both audial, transcranial, and haptic frequency can also be modulated in addition to intensity or held as a constant as presented here; all in accordance with configuration guidelines.

It should be appreciated that the relative terms of low, medium, high are relative to each other and their range of modulation is also set in accordance with configuration guidelines. Furthermore, in certain embodiments, additional modulation levels are employed like very low and very high for example.

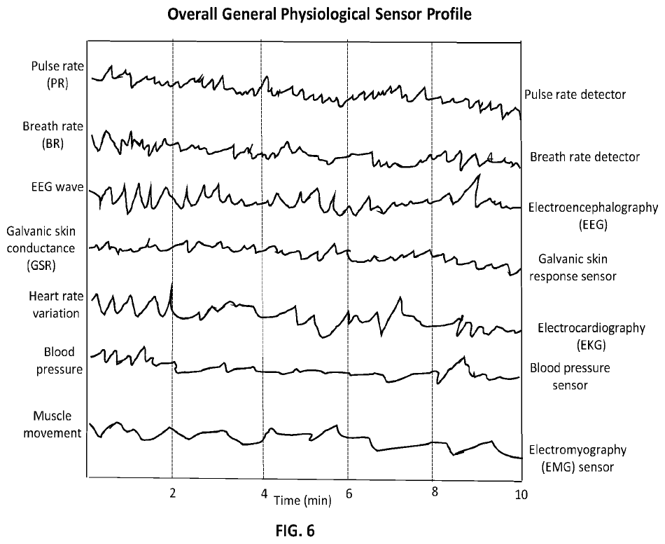

FIG. 6 is an overall physiological profile of the user experiencing the guided ocean experience of FIG. 5 as captured by the set of sensors depicted in FIG. 1. As shown, the physiological profile depicts pulse rate, breath rate, EEG wave output, galvanic skin conductance, hear rate variation, blood pressure, and muscle movement throughout the duration of the guided experience. A median value of each output is used as the basis for evaluating if each indicator is within the expected range to achieve the target value or status by the end of the stimulation session.

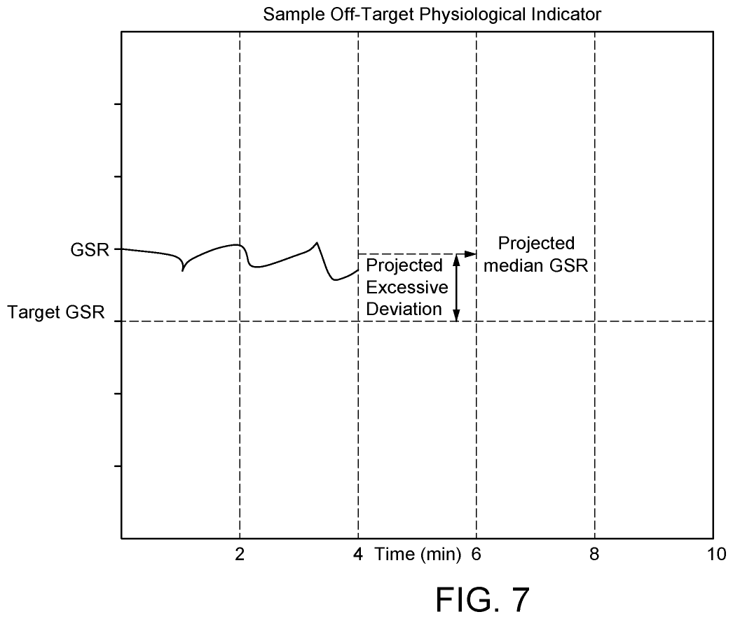

FIG. 7 depicts a median galvanic skin response that is outside of a deviation threshold of median (GSR) that will fail to achieve a target value. Accordingly, the system takes corrective action to reduce the deviation to an acceptable margin of error. It should be appreciated that such monitoring of the projected physiological indicators is implemented for all indicators the user has engaged and that GSR is discussed by way of example only.

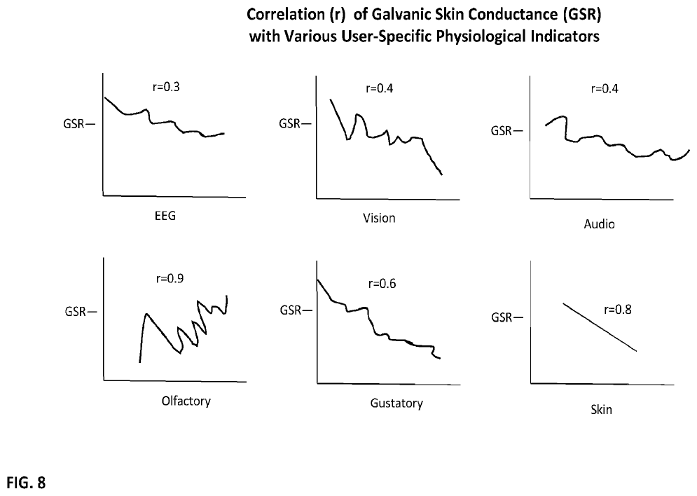

FIG. 8 depicts of series of Pearson correlation coefficients (r) between galvanic skin response and various stimulation types derived from known correlations, user profile, or the combination of both. Such correlations are employed to modify the stimulation scheme to achieve the target value GSR of FIG. 7. The correlations are stored in system database 254 depicted in FIG. 3A.

Known correlation data between various physiological factors, user profile data and experience history are employed either independently or in combination to adjust the stimulation scheme of GSR.

As shown, GSR has a correlation (r) with EEG of 0.3, with visual stimulation of 0.4, with audio of 0.4, with olfactory stimulation of 0.2, with gustatory stimulation of 0.6, and with haptic stimulation of 0.8. Pearson's r is derived from a user profile data, or when unavailable from correlations that have been generally established by those skilled in the art. Pearson's r is calculated as the product-moment correlation coefficient and it should be appreciated that other coefficient factors are employed in certain embodiments. Other correlation factors include, inter alia, intraclass correlation coefficient ICC, Spearman's ρ, Kendall's τ, Goodman and Kruskal's γ, or Somers' D, for example.

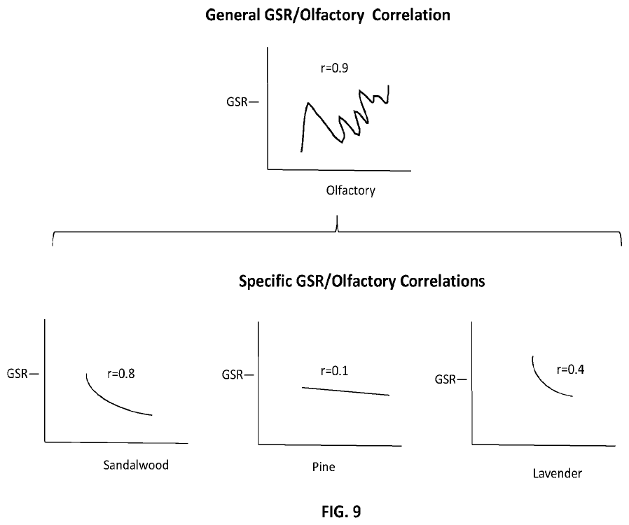

FIG. 9 depicts correlations between galvanic skin response and specific olfactory stimulation stimulations. As shown, GSR has a general correlation (r) with olfactory stimulation of 0.2, and specific correlation r of 0.8 for sandalwood, 0.1 for pine, and 0.4 for mint, with gustatory stimulation of 0.6 having a specific correlation with sweet taste of 0.7 and salty taste of 0.4.

In a certain embodiment, the system uses these sample GSR correlation values to derive a weighted average given by a proportional fraction of the sum of the values. For example, the sum of the correlations values (EEG) of 0.3+(visual) 0.4+(audial) 0.4+(olfactory) 0.2+(gustatory) 0.6+(haptic) 0.8=2.7. Analogously, a weighted value is applied to olfactory correlation based on collective, specific correlated value of (Sandalwood) 0.8+(Pine) 0.1+(Mint) 0.4=1.3. Analogously, a weighted value is applied to gustatory correlation based on a collective specific correlated value of sweet taste 0.4+salty taste 0.6=1.1. Following is a summary of the weighed sensory stimulations employed to adjust the stimulation scheme to achieve the target GSR for a certain embodiment.

Weighted Correlations with GSR

| | | | Olfactory | | | | | |
|---|---|---|---|---|---|---|---|---|
| | | | 0.07 | | | Gustatory | | |
| | | | Sandal- | | | 0.22 | | Hap- |
| EEG | Visual | Audial | wood | Pine | Mint | Sweet | Salty | tic |
| 0.11 | 0.15 | 0.15 | 0.62 | 0.08 | 0.30 | 0.37 | 0.63 | 0.30 |
| 0.110 | 0.150 | 0.150 | 0.040 | 0.006 | 0.021 | 0.081 | 0.139 | 0.300 |

It should be noted that the user preference for mint over pine finds expression in the above table of weighted correlations. Analogously, user preferences weight correlations between other physiological identifiers also.

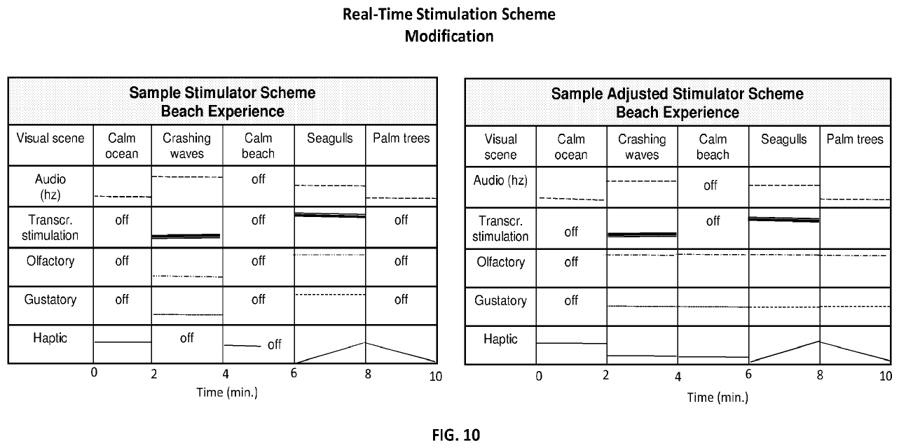

FIG. 10 depicts an adjusted stimulator scheme of the guided beach experience in which the above noted correlations between GSR and various form of olfactory stimulation define a modified stimulation scheme to achieve the target GSR. The adjusted scheme is depicted together with the previous stimulation scheme to provide context in view of the key set forth in FIG. 5; the arrows identify the modified stimulation. As shown in the adjusted scheme, light-concentration pine scent is adjusted to high-concentration sandalwood scent stimulation at two minutes and remains throughout the experience.

Low-concentration salt stimulation is increased to a medium-concentration at two minutes until six minutes and then reverts to medium-concentration sweet taste at and is adjusted high-concentration salt taste at eight minutes until the end of the experience.

Haptic simulation is augmented with low-intensity haptic simulation at two minutes and five minutes.

It should be appreciated that although a stimulation scheme has been modified to achieve a target GSR by way of example, similar schemes may be employed to achieve other physiological targets based on their respective correlations. It should be noted that posture can also contribute to a relaxion and is quantified through gyro sensor feedback. Furthermore, correlations can be time depend and the system is configurable to adjust a stimulation scheme accordingly.

It should be appreciated that the system is operative to modify one or more of the stimulations suddenly or progressively, in accordance with the nature of the activity the correlation factors, user preferences, and user responsiveness as set forth in his profile.

It should be noted that in situations in which there are various paths to achieve the target indicator values, the path minimizing modification of stimulator output is selected, in a certain configuration. For instance, if a significant modification in either smell, taste, or transcranial stimulation will be effective, whereas a smaller modification in all three stimulators will also achieve the desired target, then a smaller modification in all three stimulators will be implemented as the preferred modification.

It should be noted that physiological indicator targets are directed to indicator status levels that are relative levels set in accordance with user profiles. As noted above, absolute physiological indicator values are rated for each individual. Following are configuration options for modifying stimulation schemes to ensure achievement of user-specific, target physiological status levels, according to an embodiment.

Real-Time Feedback
In a first configuration, the stimulation modification scheme is implemented in near real-time. Stimulator parameters are modified in accordance with user responsiveness and the above-noted correlation data through machine learning techniques directed to identifying schemes having a threshold probability of achieving the target values.

Extension of Administration Time
In a second configuration, the stimulation modification scheme is implemented by extending administration time of existing a stimulation scheme. This can be implemented by either extending one or more stimulation sequences or rerunning the scheme upon its completion.

Creation of a Different Stimulation Scheme
In a third configuration, the stimulation modification scheme is implemented by constructing a new upgraded scheme characterized by either a different primary stimulator or fewer secondary stimulators accompany the existing primary stimulator. This is solution is not generally implemented in real time and would be administered in the next guided experience.

It should be appreciated that all of the above options employ machine learning techniques applied to user and demographic data to define the most effective stimulation scheme. Relevant machine learning techniques that can be employed include Bayesian networks, artificial neural networks, and other techniques providing analogous functionality.

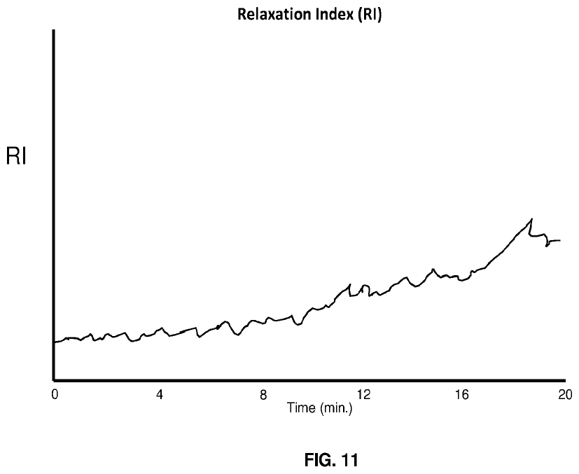

FIG. 11 depicts a plot of relation index (RI) a function of time, according to an embodiment. The RI is generated as a weighted sum of the each of the captured median physiological values and is a metric that may be used to measure and evaluate overall effectiveness of a guided meditative experience.

The full-sensory, guided meditation system embodies an advance over conventional meditation systems by engaging multiple senses to create a profoundly more immersive, meditative experience than possible with currently existing systems employing only one or two senses. Furthermore, the system embodies an additional advance over current meditative systems through autonomously personalizing the stimulation sequence to most effective engage the user in accordance with his preferences, needs, and real-time physiology.

The full-sensory, guided meditation system has application for many segments of society like medical patients needing to relax, soldiers needing to relax, students wanting to relax, and laborers who want to relax, just to name a few.

It should be appreciated that embodiments formed from combinations of features disclosed within different embodiments are included in the scope of the present invention.

While certain features of the invention have been illustrated and described herein, modifications, substitutions, and equivalents are included within the scope of the invention.

What is claimed is:

1. A method for providing full-sensory guided meditation performed on a computer having a processor, memory, and one or more code sets stored in the memory and executed in the processor, the method comprising:
administering a sensory stimulation scheme to at least one user, the stimulation scheme characterized by a themed set of sensory stimulations of one primary stimulation and a plurality of secondary stimulations; and
capturing a plurality of physiological indicators of the user during administration of the sensory stimulation scheme.

2. The method of claim 1, wherein the primary stimulation is implemented as visual imagery or audial content.

3. The method of claim 1, wherein the plurality of secondary stimulations is implemented as two or more stimulations selected from the group consisting of audial stimulation, transcranial stimulation, olfactory stimulation, gustatory stimulation, haptic stimulation, and all combinations thereof.

4. The method of claim 3, wherein transcranial stimulation is implemented as transcranial direct-current stimulation or transcranial magnetic stimulation.

5. The method of claim 1, wherein the plurality of physiological indicators is implemented as two or more indicators selected from the group consisting of breathing rate, pulse rate, heart variation, electroencephalography (EEG) feedback, blood pressure, galvanic skin conductance, muscle movement, and combinations thereof.

6. The method of claim 1, further comprising modifying the sensory stimulation scheme in accordance with at least one of the plurality of the physiological indicators.

7. The method of claim 1, further comprising defining the sensory stimulation scheme in accordance with user preferences received from the user.

8. The method of claim 7, further comprising defining the sensory stimulation scheme in accordance with physiological indicators shared by the user and a population having two or more common demographic features.

9. The method of claim 1, further comprising defining the sensory stimulation scheme in accordance with physiological indicators shared by the user and a population having three or more common demographic features.

10. The method of claim 1, further comprising outputting the physiological indicators to an output device or a dedicated user profile data base.

11. A full-sensory, guided-meditation system, the system comprising:
a set of three or more sensory stimulators of one primary stimulator and a plurality of secondary stimulators;
a computer configured to:
administer a sensory stimulation scheme to at least one user, the stimulation scheme characterized by a themed set of sensory stimulations of one primary stimulation and a plurality of secondary stimulations; and
a set of physiological sensors configured to capture a plurality of physiological indicators of the user during administration of the sensory stimulation scheme.

12. The system of claim 11, wherein the plurality of secondary stimulators is selected from a group consisting of a video display, ear phones, an olfactory stimulator, a gustatory stimulator, a haptic stimulator, a transcranial stimulator, and all combinations thereof.

13. The system of claim 12, wherein the set of a plurality of secondary stimulators contains ear phones, an olfactory stimulator, and a gustatory stimulator.

14. The system of claim 12, wherein the transcranial stimulator is implemented as a transcranial direct-current stimulator or a transcranial magnetic stimulator.

15. The system of claim 11, wherein the set of physiological sensors includes a plurality of sensors selected from the group consisting of a breathing rate sensor, a pulse rate sensor, a blood pressure sensor, a galvanic skin conductance sensor, a muscle movement sensor, a heart variation sensor, an electroencephalograph, and combinations thereof.

16. The system of claim 15, wherein the set of physiological sensors contains a breathing rate sensor, a pulse rate sensor, and a blood pressure sensor.

17. The system of claim 11, wherein the computer is further configured to modify the sensory stimulation scheme in accordance with at least one of the physiological indicators captured by the set of sensors from the user.

18. The system of claim 11, wherein the computer is further configured to define the sensory stimulation scheme in accordance with user preferences received from the user.

19. The system of claim 18, wherein the computer is further configured to define the sensory stimulation scheme in accordance with a population having two or more common demographic features with the user.

20. The system of claim 11, wherein the computer is further configured to output the physiological indicators to an output device or a dedicated user profile data base.
